(19) United States
(12) Patent Application Publication
(10) Pub. No.: US 2018/0110787 A1
(43) Pub. Date: Apr. 26, 2018

(54) Treatment for Idiopathic Pulmonary Fibrosis and Chronic Obstructive Pulmonary Disease

(71) Applicants: Buck Institute for Research on Aging, Novato, CA (US); Unity Biotechnology, Inc., Brisbane, CA (US)

(72) Inventors: Remi-Martin Laberge, San Francisco, CA (US); Nathaniel David, Brisbane, CA (US)

(21) Appl. No.: 15/808,417

(22) Filed: Nov. 9, 2017

Related U.S. Application Data
(62) Division of application No. 15/675,171, filed on Aug. 11, 2017.
Application No. 15/675,171 is a continuation-in-part of application No. 15/611,589, filed on Jun. 1, 2017.
Application No. 15/611,589 is a continuation of application No. PCT/US16/16894, filed on Feb. 5, 2016.
(60) Provisional application No. 62/113,227, filed on Feb. 6, 2015.

(51) Int. Cl. A61K 31/635; A61K 9/12; A61K 9/00

(52) U.S. Cl. CPC A61K 31/635; A61K 9/0073; A61K 9/12

(57) ABSTRACT

A library of heterocyclic compounds has been screened to identify particular compounds that have high inhibitory capacity for the Bcl family of regulatory proteins. Compounds identified as Bcl antagonists have been further screened to select pharmaceutical agents with both high potency and high specificity for eliminating senescent cells in comparison with replicative or quiescent cells of the same tissue type. Particular structures are identified in this disclosure that eliminate senescent cells with an EC₅₀ in the nanomole range and a specificity around or above 100-fold. In accordance with this invention, heterocyclic compounds provided in this disclosure can be formulated for the treatment of a range of age-related conditions caused or mediated by senescent cells. Such conditions are exemplified by ophthalmic conditions, pulmonary conditions, and osteoarthritis.

### FIELD OF THE INVENTION

The invention relates generally to the treatment of conditions that are mediated by senescent cells. In particular, it provides a family of Bcl inhibitors that are effective for eliminating such cells and are suitable for formulating as medicaments for use in clinical medicine.

### BACKGROUND

Senescent cells accumulate in tissues and organs of individuals as they age and are found at sites of age-related pathologies. Senescent cells are believed important to inhibiting proliferation of dysfunctional or damaged cells and particularly to constraining development of malignancy (Campisi, Curr. Opin. Genet. Dev. 21: 107-12 (2011); Campisi, Trends Cell Biol. 11:S27-31 (2001); Prieur et al, Curr. Opin. Cell Biol. 20: 150-55 (2008)). Nevertheless, the presence of senescent cells in an individual may contribute to aging and aging-related dysfunction (Campisi, Cell 120:513-22 (2005)). Senescent cells have been causally implicated in certain aspects of age-related decline in health and in the pathophysiology of several age-related diseases.

Inhibitors of the Bcl-2 pro-apoptotic pathway that may be suitable for killing cancer cells or senescent cells are presented in pre-grant publications US 20170056421 A1 (U. Arkansas), 20170119789 (Buck Institute), US 20160339019 (Unity Biotechnology et al.), 2016/0122758 (Yeda Research and Development), WO2015/116735 (Mayo Foundation), WO 2016/014625 (U. Arkansas), WO 2016/127135 (Unity Biotechnology), and in issued U.S. Pat. Nos. 8,691,184; 9,096,625; and 9,403,856 (U. Michigan).

### SUMMARY OF THE INVENTION

To develop this invention, a library of heterocyclic compounds was screened to identify particular compounds that have high inhibitory capacity for the Bcl family of regulatory proteins. compounds identified as Bcl antagonists were further screened for compounds having a high potency and specificity for eliminating senescent cells in comparison with replicative cells of the same tissue type. Particular structures were identified that eliminate senescent cells with an EC₅₀ in the nanomole range and a specificity of over 100-fold.

The effectiveness of the chosen compounds could not be predicted on the basis of Bcl binding by itself. Some compounds having high Bcl binding were not senolytic at a level that was clinically useful. The three-tiered screening test is included in this description as part of the invention.

Included in this invention is a method of selectively removing senescent cells from a cell population or tissue. This is done by contacting the cell population or tissue with a compound having a chemical structure that comprises Formula VI or Formula VII as shown below, or a phosphorylated form thereof

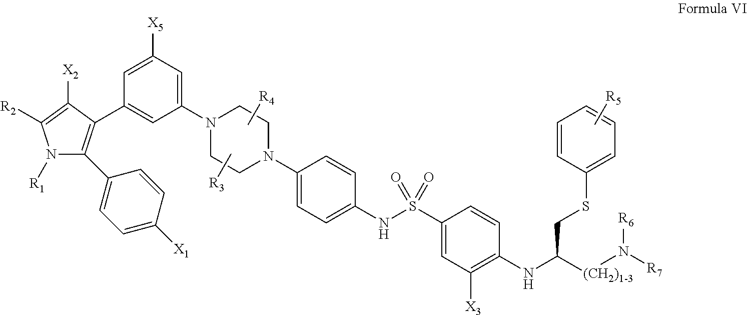

wherein:
   R₁ and R₂ are independently C₁ to C₄ alkyl;
   R₃, R₄ and R₅ are independently —H or —CH₃;
   R₆ and R₇ are independently alkyl or heteroalkyl, and are optionally cyclized;
   X₁ is —F, —Cl, —Br, or —OCH₃;
   X₂ is —SO₂R′ or —CO₂R′, where R′ is —H, —CH₃, or —CH₂CH₃;
   X₃ is —SO₂CF₃ or —NO₂; and
   X₅ is —F, —Br, —Cl, —H, or —OCH₃;
or alternatively, wherein:
   R₁ and R₂ are independently C₁ to C₄ alkyl;
   R₃ and R₄ are both —H;
   R₅ is —H or —CH₃;
   R₆ and R₇ are independently alkyl or heteroalkyl, and are optionally cyclized
      in the manner shown in Formula VII;
   X₁ is —F or —Cl;
   X₂ is —SO₂R′ or —CO₂R′, where R′ is —H, —CH₃, or —CH₂CH₃;
   X₃ is —SO₂CF₃ or —NO₂; and
   X₅ is —F or —H;

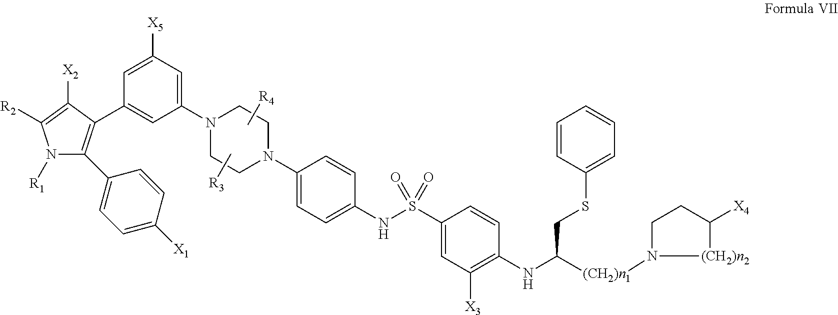

wherein:
   R₁ and R₂ are independently C₁ to C₄ alkyl;
   R₃ and R₄ are independently —H or —CH₃;
   X₁ is —F, —Cl, —Br, or —OCH₃;
   X₂ is —SO₂R′ or —CO₂R′, where R′ is —H, —CH₃, or —CH₂CH₃;
   X₃ is —SO₂CF₃ or —NO₂;
   X₄ is —OH or —COOH;
   X₅ is —F —Cl or —H; and
   n₁ and n₂ are independently 1, 2, or 3.

The cell population or tissue can be contacted with the compound in vitro, or at or around a disease site in need of treatment, such as ophthalmic tissue, in an osteoarthritic joint, or in pulmonary tissue. Typically, the compound is administered in an amount less than an amount that would be effective for removing cancer cells from the cell population or tissue.

Also included in this invention are methods of treating age related conditions such as an ophthalmic condition by administering in or around the site of the disease a pharmaceutical composition that includes a compound having a chemical structure that comprises Formula VI or VII.

The invention further provides pharmaceutical compositions, and unit doses of such compositions that contain an amount of the compound shown in Formula VI or Formula VII. The composition contains a formulation of the compound suitable for local administration in or around the site of a condition mediated by senescent cells. Formulation of the composition and the amount of the compound in the unit dose configure the composition to be effective in treating the site by decreasing the severity of one or more signs or symptoms of the condition when administered to the site as a single dose. The composition or dose is typically packaged with information for administration or use of the composition for treatment of an age-related condition.

Also provided is a screening method of identifying senolytic compounds suitable for use in treating conditions mediated by senescent cells. The method comprises screening the library for binding to any one or more of the Bcl isoforms, particularly Bcl-xL, Bcl-2, and/or Bcl-w in any combination, and selecting compounds that have sufficient affinity for the target regulator protein(s)—below 1, 0.5 or 0.3 nanomolar, in order of increasing preference. The method further comprises screening the library for an ability to kill senescent cells (such as senescent fibroblasts) at low concentration (high potency)—having an EC₅₀ below 1, 0.1, or 0.01 micromolar, in order of increasing preference. The method further comprises screening the library for high specificity or selectivity for killing senescent cells compared with non-senescent cells of the same tissue type—having a specificity index (SI) above 10, 50, or 200 in order of increasing preference. The compounds ultimately selected have high affinity for at least one of the Bcl isoforms, high potency for killing senescent cells, and high specificity. The steps of the screening method can be performed in any workable order.

Certain features of the invention are referred to in the appended claims. Other features are referred to in the description that follows. The features described in this disclosure can be selected for use as part of a pharmaceutical composition, a method of use in vitro, or a method of medical therapy in any operable combination.

### DRAWINGS

FIGS. 1A, 1B, and 1C show nine particular compounds selected from a library on the basis of binding to Bcl-2 or Bcl-xL.

FIGS. 2A, 2B, and 2C show quantitative binding affinity of the nine compounds to Bcl isoforms Bcl-xL, Bcl-2, and Bcl-w, respectively. Each of the compounds for which the data is shown are identified according to their designated BM number.

FIGS. 3A, 3B, and 3C shows how effective compounds were compared structurally to determine what substructures contribute to the desired properties of the compounds.

FIG. 4 shows binding affinity to Bcl isoforms, and the effective concentration (EC₅₀) for killing senescent fibroblasts (SnCs) in culture.

### DETAILED DESCRIPTION

This invention provides a family of heterocyclic Bcl-xL inhibitors and related analogs that are specially effective for removing senescent cells and treating age related conditions. By way of illustration, such conditions include conditions of the eye, pulmonary conditions, osteoarthritis, and atherosclerosis. The invention also includes treatment of other age-related conditions, exemplified by but not limited to those described in the sections that follow and in the previous disclosures to which this application claims priority.

1. Selection of Compounds from a Compound Library

The compounds according to Formula VI and Formula VII shown above were selected from a compound library as having particularly beneficial properties (potency and selectivity) for purposes of killing senescent cells and treating senescent-related conditions.

The library from which the compounds were selected include compounds having the general formula as shown in Formula I.

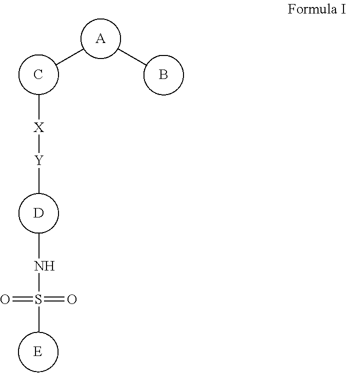

wherein: A is absent, alkaline, alkenylene, alkynylene, arylene, cycloalkylene, heteroarylene, heterocycloalkylene, aminocarbonyl, alkoxycarbonyl, an ether linkage, a sulfonyl linkage, a carbamate linkage, a carbonate linkage, an amide linkage, a urea linkage, or an ester linkage, any of which is substituted or unsubstituted: B is alkyl, alkenyl, alkynyl, aryl, cycloalkyl, heteroaryl, heterocycloalkyl, acyl, aminocarbonyl, alkoxycarbonyl, alkoxy, cycloalkyloxy, heterocycloalkyloxy, sulfonyl, carbamate, carbonate, amide, amine, urea, or ester; any of which is substituted or unsubstituted, or H; E is alkyl, alkenyl, alkynyl, aryl, cycloalkyl, heteroaryl, heterocycloalkyl, acyl, aminocarbonyl, alkoxycarbonyl, alkoxy, cycloalkyloxy, heterocycloalkyloxy, sulfonyl, carbamate, carbonate, amide, amine, urea, or ester; any of which is substituted or unsubstituted, or H; C is absent, alkaline, alkenylene, alkynylene, arylene, cycloalkylene, heteroarylene, heterocycloalkylene, aminocarbonyl, alkoxycarbonyl, an ether linkage, a sulfonyl linkage, a carbamate linkage, a carbonate linkage, an amide linkage, a urea linkage, or an ester linkage, any of which is substituted or unsubstituted; D is absent, alkaline, alkenylene, alkynylene, arylene, cycloalkylene, heteroarylene, heterocycloalkylene, aminocarbonyl, alkoxycarbonyl, an ether linkage, a sulfonyl linkage, a carbamate linkage, a carbonate linkage, an amide linkage, a urea linkage, or an ester linkage, any of which is substituted or unsubstituted; X and Y are independently absent, 0, S, CO, SO₂, SO, PO₃H, NR′, BR′, PR′, POR′, alkaline, cycloalkylene, alkenylene, cycloalkenylene, alkynylene, arylene, heteroarylene, or heterocyclylene, any of which is substituted or unsubstituted, or any combination thereof; or X and Y are members of a ring; each R′ is independently alkyl, alkenyl, alkynyl, aryl, aralkyl, cycloalkyl, heteroaryl, heterocycloalkyl, acyl, aminocarbonyl, alkoxycarbonyl, alkoxy, cycloalkyloxy, heterocycloalkyloxy, sulfonyl, cycloalkeny, or H, or a pharmaceutically-acceptable salt thereof.

The library includes compounds according to Formula III, IV or V, shown here in a phosphorylated form.

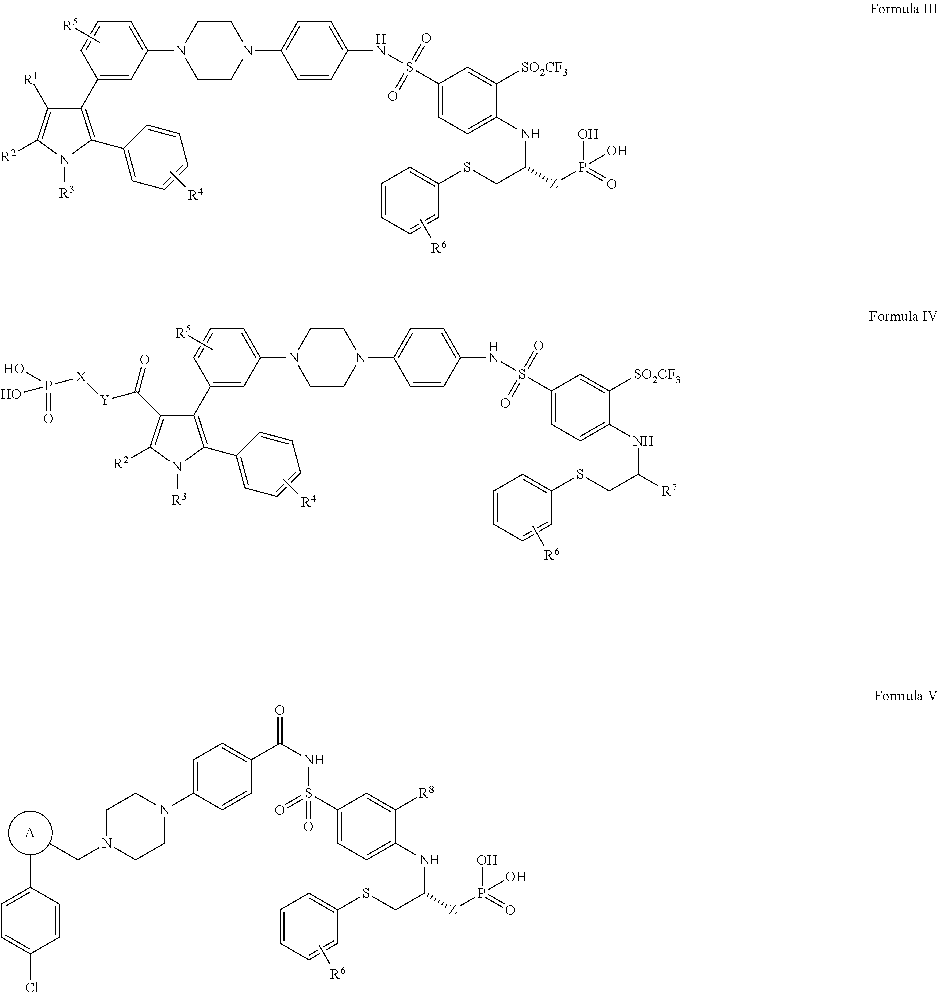

wherein Z is (CH₂)ₙ—N(R^{a})₂ or

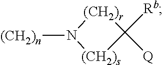

and wherein Q is OH, alkoxy, O(CH₂)₁₋₃, NHR^{c}, NR^{c}(C₁₋₃alkylene), NR^{c}(alkaline), OC(═O)(C₁₋₃alkylene), OC(═O)(alkaline), C(═O)O(alkaline), C(═O)O(C₁₋₃alkylene), NHC(═O)(alkaline), NHC(═O)(C₁₋₃alkylene), C(═O)NHR^{c}, C(═O)NH(alkaline), C(═O)NH(C₁₋₃alkylene); R^{c} is hydrogen, CN, NO₂, CF₃, halo, hydroxyalkyl, optionally substituted alkoxy, optionally substituted alkyl, optionally substituted cycloalkyl, optionally substituted alkenyl, optionally substituted cycloalkenyl, optionally substituted alkynyl, optionally substituted aryl, optionally substituted heteroaryl, or optionally substituted heterocycloalkyl; n, r, and s are independently 1, 2, 3, 4, 5, or 6. X, Z, the A ring, R₁, R₂, R₃, R₄, R₅, R₆, R₇, R₈, R′, R″, R′″, R^{a} and R^{b} according to these formulae are defined in priority application PCT/US2016/016894 (WO 2016/127135).

### Screening of Compounds for Apoptosis Factor Binding Activity and Senolysis

Compounds can be screened on a molecular level for their ability to inhibit the binding between Bcl-2 or Bcl-xL and their respective cognate ligand. Example 1 below provides an illustration of a homogeneous assay for this purpose.

Alternatively or in addition, compounds can be screened for biological activity in an assay using senescent cells. Cultured cells are contacted with the compound, and the degree of cytotoxicity or inhibition of the cells is determined. The ability of the compound to kill or inhibit senescent cells can be compared with the effect of the compound on normal cells that are freely dividing at low density, and normal cells that are in a quiescent state at high density. Example 2 provides an illustration using the human lung fibroblast IMR90 cell line.

### Ophthalmic Conditions

Diseases of the eye that can be treated according to this invention include presbyopia, macular degeneration (including wet or dry AMD), diabetic retinopathy, and glaucoma.

Macular degeneration is a neurodegenerative condition that can be characterized as a back-of-the-eye disease, It causes the loss of photoreceptor cells in the central part of retina, called the macula. Macular degeneration can be dry or wet. The dry form is more common than the wet, with about 90% of age-related macular degeneration (AMD) patients diagnosed with the dry form. The wet form of the disease can lead to more serious vision loss. Age and certain genetic factors and environmental factors can be risk factors for developing AMD. Environmental factors include, for example, omega-3 fatty acids intake, estrogen exposure, and increased serum levels of vitamin D. Genetic risk factors can include, for example, reduced ocular Dicer1 levels, and decreased micro RNAs, and DICER1 ablation.

Dry AMD is associated with atrophy of the retinal pigment epithelium (RPE) layer, which causes loss of photoreceptor cells. The dry form of AMD can result from aging and thinning of macular tissues and from deposition of pigment in the macula. With wet AMD, new blood vessels can grow beneath the retina and leak blood and fluid. Abnormally leaky choroidal neovascularization can cause the retinal cells to die, creating blind spots in central vision. Different forms of macular degeneration can also occur in younger patients. Non-age related etiology can be linked to, for example, heredity, diabetes, nutritional deficits, head injury, or infection.

The formation of exudates, or “drusen,” underneath the Bruch's membrane of the macula is can be a physical sign that macular degeneration can develop. Symptoms of macular degeneration include, for example, perceived distortion of straight lines and, in some cases, the center of vision appears more distorted than the rest of a scene; a dark, blurry area or “white-out” appears in the center of vision; or color perception changes or diminishes.

Another back-of-the-eye disease is diabetic retinopathy (DR). According to Wikipedia, the first stage of DR is non-proliferative, and typically has no substantial symptoms or signs. NPDR is detectable by fundus photography, in which microaneurysms (microscopic blood-filled bulges in the artery walls) can be seen. If there is reduced vision, fluorescein angiography can be done to see the back of the eye. Narrowing or blocked retinal blood vessels can be seen clearly and this is called retinal ischemia (lack of blood flow). Macular edema in which blood vessels leak their contents into the macular region can occur at any stage of NPDR. The symptoms of macular edema are blurred vision and darkened or distorted images that are not the same in both eyes. Ten percent (10%) of diabetic patients will have vision loss related to macular edema. Optical Coherence Tomography can show the areas of retinal thickening (due to fluid accumulation) of macular edema.

In the second stage of DR, abnormal new blood vessels (neovascularisation) form at the back of the eye as part of proliferative diabetic retinopathy (PDR); these can burst and bleed (vitreous hemorrhage) and blur the vision, because these new blood vessels are fragile. The first time this bleeding occurs, it may not be very severe. In most cases, it will leave just a few specks of blood, or spots floating in a person's visual field, though the spots often go away after few hours. These spots are often followed within a few days or weeks by a much greater leakage of blood, which blurs the vision. In extreme cases, a person may only be able to tell light from dark in that eye. It may take the blood anywhere from a few days to months or even years to clear from the inside of the eye, and in some cases the blood will not clear. These types of large hemorrhages tend to happen more than once, often during sleep.

On funduscopic exam, a doctor will see cotton wool spots, flame hemorrhages (similar lesions are also caused by the alpha-toxin of Clostridium novyi), and dot-blot hemorrhages.

Presbyopia is an age-related condition where the eye exhibits a progressively diminished ability to focus on near objects as the speed and amplitude of accommodation of a normal eye decreases with advancing age. Loss of elasticity of the crystalline lens and loss of contractility of the ciliary muscles can cause presbyopia. Age-related changes in the mechanical properties of the anterior lens capsule and posterior lens capsule suggest that the mechanical strength of the posterior lens capsule decreases significantly with age.

The laminated structure of the capsule of the eye also changes and can result, at least in part, from a change in the composition of the tissue. The major structural component of the lens capsule is basement membrane type IV collagen that is organized into a three-dimensional molecular network. Type IV collagen is composed of six homologous α chains (α1-6) that associate into heterotrimeric collagen IV protomers with each comprising a specific chain combination of α112, α345, or α556. Protomers share structural similarities of a triple-helical collagenous domain with the triplet peptide sequence of Gly-X-Y, ending in a globular C-terminal region termed the non-collagenous 1 (NC1) domain. The N-termini are composed of a helical domain termed the 7S domain, which is also involved in protomer-protomer interactions.

Collagen IV can influence cellular function and tissue stabilization. Posterior capsule opacification (PCO) develops as a complication in approximately 20-40% of patients in subsequent years after cataract surgery. PCO results from proliferation and activity of residual lens epithelial cells along the posterior capsule in a response akin to wound healing. Growth factors, such as fibroblast growth factor, transforming growth factor 0, epidermal growth factor, hepatocyte growth factor, insulin-like growth factor, and interleukins IL-1 and IL-6, can also promote epithelial cell migration. In vitro, collagen IV can promote adherence of lens epithelial cells. Adhesion of the collagen IV, fibronectin, and laminin to the intraocular lens can inhibit cell migration and can reduce the risk of PCO.

Compounds provided by this disclosure can slow the disorganization of the type IV collagen network, decrease or inhibit epithelial cell migration and can also delay the onset of presbyopia or decrease or slow the progressive severity of the condition. They can also be useful for post-cataract surgery to reduce the likelihood of occurrence of PCO.

Another condition treatable according to this invention is glaucoma. Normally, clear fluid flows into and out of the front part of the eye, known as the anterior chamber. In individuals who have open/wide-angle glaucoma, the clear fluid drains too slowly, leading to increased pressure within the eye. If left untreated, the high pressure in the eye can subsequently damage the optic nerve and can lead to complete blindness. The loss of peripheral vision is caused by the death of ganglion cells in the retina. The effect of a therapy on inhibiting progression of glaucoma can be monitored by automated perimetry, gonioscopy, imaging technology, scanning laser tomography, HRT3, laser polarimetry, GDX, ocular coherence tomography, ophthalmoscopy, and pachymeter measurements that determine central corneal thickness.

### Cardiovascular Conditions and Atherosclerosis

Cardiovascular conditions that can potentially be treated according to this invention include arrhythmia, atherosclerosis, cardiomyopathy, congestive heart failure, coronary artery disease (CAD), carotid artery disease, endocarditis, heart attack, coronary thrombosis, myocardial infarction [MI], high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease, peripheral artery disease (PAD), cardiac stress resistance, and stroke.

The methods for treating a cardiovascular disease that is associated with or caused by arteriosclerosis can reduce the likelihood of occurrence of high blood pressure/hypertension, angina, stroke, and heart attack, coronary thrombosis, and myocardial infarction (MI). Methods are provided by this invention for stabilizing atherosclerotic plaque(s) in a blood vessel, for example, an artery, of a subject, thereby reducing the likelihood of occurrence or delaying the occurrence of a thrombotic event, such as stroke or MI. The active compound may reduce the lipid content of an atherosclerotic plaque in a blood vessel, for example, an artery, of the subject, or increase the fibrous cap thickness by, for example, causing an increase, enhancing or promoting thickening of the fibrous cap.

Atherosclerosis is characterized by patchy intimal plaques, atheromas, that encroach on the lumen of medium-sized and large arteries; the plaques contain lipids, inflammatory cells, smooth muscle cells, and connective tissue. Atherosclerosis can affect large and medium-sized arteries, including the coronary, carotid, and cerebral arteries, the aorta and branches thereof, and major arteries of the extremities.

Methods are provided in this disclosure for inhibiting, reducing, or causing a decrease in, the formation of atherosclerotic plaques by administering a compound of this invention to a subject in need thereof. Reduction in the amount of plaque in a blood vessel, for example, an artery, can be determined, for example, by a decrease in surface area of the plaque, or by a decrease in the extent, degree, or percent of occlusion of a blood vessel, for example, an artery, which can be determined by angiography or other visualizing methods.

Also provided by this invention are methods for increasing, improving, promoting, or enhancing the stability of atherosclerotic plaques that are present in one or more blood vessels, for example, one or more arteries of a subject.

Atherosclerosis can be a hardening or furring of the arteries and is caused by the formation of multiple atheromatous plaques within the arteries. Atherosclerosis (also called arteriosclerotic vascular disease or ASVD) is a form of arteriosclerosis in which an artery wall thickens. Symptoms develop when growth or rupture of the plaque reduces or obstructs blood flow; and the symptoms can vary depending on which artery is affected. Atherosclerotic plaques can be stable or unstable. Stable plaques regress, remain static, or grow slowly, sometimes over several decades, until they can cause stenosis or occlusion. Unstable plaques are vulnerable to spontaneous erosion, fissure, or rupture, causing acute thrombosis, occlusion, and infarction long before they cause hemodynamically significant stenosis. Clinical events can result from unstable plaques, which do not appear severe on angiography; thus, plaque stabilization can be a way to reduce morbidity and mortality. Plaque rupture or erosion can lead to major cardiovascular events such as acute coronary syndrome and stroke. Disrupted plaques can have a greater content of lipid, macrophages, and have a thinner fibrous cap than intact plaques.

Atherosclerosis is a syndrome affecting arterial blood vessels due in significant part to a chronic inflammatory response of white blood cells in the walls of arteries. This is promoted by low-density lipoproteins (LDL), plasma proteins that carry cholesterol and triglycerides, in the absence of adequate removal of fats and cholesterol from macrophages by functional high-density lipoproteins (HDL). The earliest visible lesion of atherosclerosis is the “fatty streak,” which is an accumulation of lipid-laden foam cells in the intimal layer of the artery. The hallmark of atherosclerosis is atherosclerotic

Diagnosis of atherosclerosis and other cardiovascular disease can be based on symptoms, for example, angina, chest pressure, numbness or weakness in arms or legs, difficulty speaking or slurred speech, drooping muscles in face, leg pain, high blood pressure, kidney failure and/or erectile dysfunction, medical history, and/or physical examination of a patient. Diagnosis can be confirmed by angiography, ultrasonography, or other imaging tests. Subjects at risk of developing cardiovascular disease include those having any one or more of predisposing factors, such as a family history of cardiovascular disease and those having other risk factors, for example, predisposing factors including high blood pressure, dyslipidemia, high cholesterol, diabetes, obesity and cigarette smoking, sedentary lifestyle, and hypertension.

The condition can be assessed, for example, by angiography, electrocardiography, or stress test. The effects of the treatment of a compound can be analyzed by, for example, comparing symptoms of patients suffering from or at risk of cardiovascular disease that have received the treatment with those of patients without such a treatment or with placebo treatment.

### Osteoarthritis

Osteoarthritis degenerative joint disease is characterized by fibrillation of the cartilage at sites of high mechanical stress, bone sclerosis, and thickening of the synovium and the joint capsule. Fibrillation is a local surface disorganization involving splitting of the superficial layers of the cartilage. The early splitting is tangential with the cartilage surface, following the axes of the predominant collagen bundles. Collagen within the cartilage becomes disorganized, and proteoglycans are lost from the cartilage surface. In the absence of protective and lubricating effects of proteoglycans in a joint, collagen fibers become susceptible to degradation, and mechanical destruction ensues. Predisposing risk factors for developing osteoarthritis include increasing age, obesity, previous joint injury, overuse of the joint, weak thigh muscles, and genetics. Symptoms of osteoarthritis include sore or stiff joints, particularly the hips, knees, and lower back, after inactivity or overuse; stiffness after resting that goes away after movement; and pain that is worse after activity or toward the end of the day.

Compounds according to this invention can be used to reduce or inhibit loss or erosion of proteoglycan layers in a joint, reduces inflammation in the affected joint, and promotes, stimulates, enhances, or induces production of collagen, for example, type 2 collagen. The compound may causes a reduction in the amount, or level, of inflammatory cytokines, such as IL-6, produced in a joint and inflammation is reduced. The compounds can be used for treating osteoarthritis and/or inducing collagen, for example, Type 2 collagen, production in the joint of a subject. A compound also can be used for decreasing, inhibiting, or reducing production of metalloproteinase 13 (MMP-13), which degrades collagen in a joint, and for restoring proteoglycan layer or inhibiting loss and/or degradation of the proteoglycan layer. Treatment with a compound thereby may also reduce the likelihood of, inhibits, or decreases erosion, or slows erosion of the bone. The compound may be administered directly to an osteoarthritic joint, for example, intra-articularly, topically, transdermally, intradermally, or subcutaneously. The compound may also restore, improve, or inhibit deterioration of strength of a join, and reduce joint pain.

### Pulmonary Conditions

Pulmonary conditions that can be treated according to this invention include idiopathic pulmonary fibrosis (IPF), chronic obstructive pulmonary disease (COPD), asthma, cystic fibrosis, bronchiectasis, and emphysema.

COPD is a lung disease defined by persistently poor airflow resulting from the breakdown of lung tissue, emphysema, and the dysfunction of the small airways, obstructive bronchiolitis. Primary symptoms of COPD include shortness of breath, wheezing, chest tightness, chronic cough, and excess sputum production. Elastase from cigarette smoke-activated neutrophils and macrophages can disintegrate the extracellular matrix of alveolar structures, resulting in enlarged air spaces and loss of respiratory capacity. COPD can be caused by, for example, tobacco smoke, cigarette smoke, cigar smoke, secondhand smoke, pipe smoke, occupational exposure, exposure to dust, smoke, fumes, and pollution, occurring over decades thereby implicating aging as a risk factor for developing COPD.

The processes that cause lung damage include, for example, oxidative stress produced by the high concentrations of free radicals in tobacco smoke, cytokine release due to the inflammatory response to irritants in the airway, and impairment of anti-protease enzymes by tobacco smoke and free radicals, allowing proteases to damage the lungs. Genetic susceptibility can also contribute to the disease. In about 1% percent of people with COPD, the disease results from a genetic disorder that causes low level production of alpha-1-antitrypsin in the liver. Alpha-1-antitrypsin is normally secreted into the bloodstream to help protect the lungs.

Pulmonary fibrosis is a chronic and progressive lung disease characterized by stiffening and scarring of the lung, which can lead to respiratory failure, lung cancer, and heart failure. Fibrosis is associated with repair of epithelium. Fibroblasts are activated, production of extracellular matrix proteins is increased, and transdifferentiation to contractile myofibroblasts contribute to wound contraction. A provisional matrix plugs the injured epithelium and provides a scaffold for epithelial cell migration, involving an epithelial-mesenchymal transition (EMT). Blood loss associated with epithelial injury induces platelet activation, production of growth factors, and an acute inflammatory response. Normally, the epithelial barrier heals and the inflammatory response resolves. However, in fibrotic disease the fibroblast response continues, resulting in unresolved wound healing. Formation of fibroblastic foci is a feature of the disease, reflecting locations of ongoing fibrogenesis.

Subjects at risk of developing pulmonary fibrosis include, for example, those exposed to environmental or occupational pollutants, such as asbestosis and silicosis; those who smoke cigarettes; those who have a connective tissue diseases such as RA, SLE, scleroderma, sarcoidosis, or Wegener's granulomatosis; those who have infections; those who take certain medications, including, for example, amiodarone, bleomycin, busufan, methotrexate, and nitrofurantoin; those subject to radiation therapy to the chest; and those whose family member have pulmonary fibrosis.

Symptoms of COPD can include any one of shortness of breath, wheezing, chest tightness, having to clear one's throat first thing in the morning because of excess mucus in the lungs, a chronic cough that produces sputum that can be clear, white, yellow or greenish, cyanosis, frequent respiratory infections, lack of energy, and unintended weight loss. Subjects with COPD can also experience exacerbations, during which symptoms worsen and persist for days or longer. Symptoms of pulmonary fibrosis include, for example, shortness of breath, particularly during exercise; dry, hacking cough; fast, shallow breathing; gradual, unintended weight loss; fatigue; aching joints and muscles; and clubbing of the fingers or toes.

Other pulmonary conditions that can be treated by using a compound according to this condition include emphysema, asthma, bronchiectasis, and cystic fibrosis. Pulmonary diseases can also be exacerbated by tobacco smoke, occupational exposure to dust, smoke, or fumes, infection, or pollutants that contribute to inflammation.

Bronchiectasis can result from damage to the airways that causes them to widen and become flabby and scarred. Bronchiectasis can be caused by a medical condition that injures the airway walls or inhibits the airways from clearing mucus. Examples of such conditions include cystic fibrosis and primary ciliary dyskinesia (PCD). When only one part of the lung is affected, the disorder can be caused by a blockage rather than a medical condition.

The methods of this invention for treating or reducing the likelihood of a pulmonary condition can also be used for treating a subject who is aging and has loss of pulmonary function, or degeneration of pulmonary tissue. The respiratory system can undergo various anatomical, physiological and immunological changes with age. The structural changes include chest wall and thoracic spine deformities that can impair the total respiratory system compliance resulting in increased effort to breathe. The respiratory system undergoes structural, physiological, and immunological changes with age. An increased proportion of neutrophils and lower percentage of macrophages can be found in bronchoalveolar lavage (BAL) of older adults compared with younger adults. Persistent low grade inflammation in the lower respiratory tract can cause proteolytic and oxidant-mediated injury to the lung matrix resulting in loss of alveolar unit and impaired gas exchange across the alveolar membrane seen with aging. Sustained inflammation of the lower respiratory tract can predispose older adults to increased susceptibility to toxic environmental exposure and accelerated lung function decline. Oxidative stress exacerbates inflammation during aging. Alterations in redox balance and increased oxidative stress during aging precipitate the expression of cytokines, chemokines, and adhesion molecules, and enzymes. Constitutive activation and recruitment of macrophages, T cells, and mast cells foster release of proteases leading to extracellular matrix degradation, cell death, remodeling, and other events that can cause tissue and organ damage during chronic inflammation.

The effects of the treatment can be determined using techniques that evaluate mechanical functioning of the lung, for example, techniques that measure lung capacitance, elastance, and airway hypersensitivity can be performed. To determine lung function and to monitor lung function throughout treatment, any one of numerous measurements can be obtained, for example, expiratory reserve volume (ERV), forced vital capacity (FVC), forced expiratory volume (FEV) (e.g., FEV in one second, FEV1), FEV1/FEV ratio, forced expiratory flow 25% to 75%, and maximum voluntary ventilation (MVV), peak expiratory flow (PEF), slow vital capacity (SVC). Total lung volumes include total lung capacity (TLC), vital capacity (VC), residual volume (RV), and functional residual capacity (FRC). Gas exchange across alveolar capillary membrane can be measured using diffusion capacity for carbon monoxide (DLCO). Peripheral capillary oxygen saturation (SpO₂) can also be measured; normal oxygen levels are typically between 95% and 100%. An SpO₂ level below 90% suggests the subject has hypoxemia. Values below 80% are considered critical and require intervention to maintain brain and cardiac function and avoid cardiac or respiratory arrest.

Pharmaceutical Formulations and their Use

A pharmaceutical composition of according to this invention typically includes a pharmaceutically active agent described above, in combination with other components, such as carriers, stabilizers, diluents, dispersing agents, suspending agents, thickening agents, and/or excipients. Physiologically-acceptable carriers can be chosen to facilitate uptake or duration of action at or around the treatment site. The formulation can be adapted depending upon the route of administration chosen. In preparing pharmaceutical compositions, the reader may refer to Remington: The Science and Practice of Pharmacy, Nineteenth Ed (Easton, Pa.: Mack Publishing Company, 1995), and other suitable reference materials.

Pharmaceutical compositions can be administered in therapeutically-effective amounts as pharmaceutical compositions either by systemic or by local administration, including, for example, intravenous, subcutaneous, intramuscular, oral, rectal, parenteral, ophthalmic, pulmonary, transdermal, vaginal, intravitreal, nasal, and topical administration.

Compositions of the invention can be packaged as a kit, including written instructions on the administration/use of the composition for particular conditions. Pharmaceutical compositions can be packaged as unit dosage forms suitable for single administration of precise dosages.

Use of Bcl inhibitors according to this invention for killing senescent cells or treating senescent-related conditions typically requires a dose, a frequency of administration, and an overall burden that is considerably less (100-fold or better) less than what is required to treat a cancer or hyperproliferative condition at or around the same site or in the same tissue type. For example, for treatment of senescent related conditions, the compound is administered as a single dose or in a plurality of treatment cycles, wherein each treatment cycle independently comprises a treatment course of from one day to 3 months followed by a non-treatment interval of at least 2 weeks.

### Definitions

A “senescent cell” is generally thought to be derived from a cell type that typically replicates, but as a result of aging or other event that causes a change in cell state, can no longer replicate. It remains metabolically active and commonly adopts a senescence associated secretory phenotype (SASP). The nucleus of senescent cells is often characterized by senescence-associated heterochromatin foci and DNA segments with chromatin alterations reinforcing senescence. Without implying any limitation on the practice of what is claimed in this disclosure that is not explicitly stated or required, the invention is premised on the hypothesis that senescent cells cause or mediate certain conditions associated with tissue damage or aging. For the purpose of practicing aspects of this inveniton, senescnet cells can be identified as experssing at least one marker selected from p16, senescence-associated β-galactosidase, and lipofuscin; sometimes two or more of these markers, and optionally other markers of SASP such as interleukin 6.

A “senescence associated” disease, disorder, or condition is a physiological condition that presents with by one or more symptoms or signs, wherein a subject having the condition needs or would benefit from a lessening of such symptoms or signs. The condition is senescence associated if it occurs predominantly in people over 65 years of age, or if it is caused or mediated in part by senescent cells. Lists of senescence associated disorder that can potentially be treated or managed using the methods and products taught in this disclosure include but are not limited to those discussed in this disclosure and the previous disclosures to which this application claims priority.

A compound is “senolytic” if it eliminates senescent cells preferentially, compared with replicative cells of the same tissue type, or quiescent cells lacking SASP markers.

A “phosphorylated” form of a compound is a compound in which one or more —OH or —COOH groups have been substituted with a phosphate group which is either —OPO₃H₂ or —CₙPO₃H₂ (where n is 1 to 4), such that the phosphate group may be removed in vivo (for example, by enzymolysis). A non-phosphorylated or dephosphorylated form has no such group.

Unless otherwise stated or required, all the compound structures referred to in the invention include conjugate acids and bases having the same structure, crystalline and amorphous forms of those compounds, pharmaceutically acceptable salts, and dissolved and solid forms thereof, including, for example, polymorphs, solvates, hydrates, unsolvated polymorphs (including anhydrates), conformational polymorphs, and amorphous forms of the compounds, as well as mixtures thereof.

Except where otherwise stated or required, other terms used in the specification have their ordinary meaning.

### INCORPORATION BY REFERENCE

Each and every publication and patent document cited in this disclosure is hereby incorporated herein by reference in its entirety for all purposes to the same extent as if each such publication or document was specifically and individually indicated to be incorporated herein by reference.

US 2016/0339019 A1 (Laberge et al.) is hereby incorporated herein by reference in its entirety for all purposes, including the use of senolytic compounds to treat various age-related conditions and formulation as medicaments. U.S. Pat. Nos. 8,691,184, 9,096,625, and 9,403,856 (Wang et al.) are hereby incorporated herein by reference in its entirety for all purposes, including the features of compounds in the Bcl library, their preparation and use.

### EXAMPLES

### Example 1: Measuring Bcl Inhibition

For senescnet cells and senescent-like cells having active Bcl activity, effective senolytic agents can be screened first by measuring binding to Bcl isoforms.

This assay uses a homogenous assay technology based on oxygen channeling that is marketed by PerkinElmer Inc., Waltham, Mass.: see Eglen et al., Current Chemical Genomics, 2008, 1, 2-10. The test compound is combined with the target Bcl protein and a peptide representing the corresponding cognate ligand, labeled with biotin. The mixture is then combined with streptavidin bearing luminescent donor beads and luminescent acceptor beads, which proportionally reduces luminescence if the compound has inhibited the peptide from binding to the Bcl protein.

Bcl-2, Bcl-xL and Bcl-W are available from Sigma-Aldrich Co., St. Louis, Mo. Biotinylated BIM peptide (ligand for Bcl-2) and BAD peptide (ligand for Bcl-xL) are described in pre-grant publication US 2016/0038503 A1. AlphaScreen® Streptavidin donor beads and Anti-6×His AlphaLISA® acceptor beads are available from PerkinElmer.

To conduct the assay, a 1:4 dilution series of the compound is prepared in DMSO, and then diluted in assay buffer. In a 96-well PCR plate, the following are combined: 10 μL peptide, 10 μL test compound, and 10 μL BCL protein. The assay plate is incubated in the dark at room temperature for 24 h. The next day, donor beads and acceptor beads are combined, and 5 μL is added to each well. After incubating in the dark for 30 minute, luminescence is measured using a plate reader, and the affinity or degree of inhibition by each test compound is determined.

### Example 2: Measuring Senolytic Activity

Ability of candidate agents to eliminate senescent cells or senescent-like cells can be measured directly, for example, in the following assay.

Human fibroblast IMR90 cells can be obtained from the American Type Culture Collection (ATCC®) with the designation CCL-186. The cells are maintained at <75% confluency in DMEM containing FBS and Pen/Strep in an atmosphere of 3% O₂, 10% CO₂, and ˜95% humidity. The cells are divided into three groups: irradiated cells (cultured for 14 days after irradiation prior to use), proliferating normal cells (cultured at low density for one day prior to use), and quiescent cells (cultured at high density for four day prior to use).

On day 0, the irradiated cells are prepared as follows. IMR90 cells are washed, placed in T175 flasks at a density of 50,000 cells per mL, and irradiated at 10-15 Gy. Following irradiation, the cells are plated at 100 μL in 96-well plates. On days 1, 3, 6, 10, and 13, the medium in each well is aspirated and replaced with fresh medium.

On day 10, the quiescent healthy cells are prepared as follows. IMR90 cells are washed, covered with 3 mL of TrypLE trypsin-containing reagent (Thermofisher Scientific, Waltham, Mass.) and cultured for 5 min until the cells have rounded up and begin to detach from the plate. Cells are dispersed, counted, and prepared in medium at a concentration of 50,000 cells per mL. 100 μL of the cells is plated in each well of a 96-well plate. Medium is changed on day 13.

On day 13, the proliferating healthy cell population is prepared as follows. Healthy IMR90 cells are washed, covered with 3 mL of TrypLE and cultured for 5 minutes until the cells have rounded up and begin to detach from the plate. Cells are dispersed, counted, and prepared in medium at a concentration of 25,000 cells per mL. 100 μL of the cells is plated in each well of a 96-well plate.

On day 14, test senolytic agents are combined with the cells as follows. A DMSO dilution series of each test compound is prepared at 200 times the final desired concentration in a 96-well PCR plate. Immediately before use, the DMSO stocks are diluted 1:200 into prewarmed complete medium. Medium is aspirated from the cells in each well, and 100 μL/well of the compound containing medium is added.

The test senolytic agents are cultured with the cells for 6 days, replacing the culture medium with fresh medium and the same compound concentration on day 17. Bcl-2 inhibitors such as those tested in the examples that follow are cultured with the cells for 3 days. The assay system uses the properties of a thermostable luciferase to enable reaction conditions that generate a stable luminescent signal while simultaneously inhibiting endogenous ATPase released during cell lysis. At the end of the culture period, 100 μL of CellTiter-Glo® reagent (Promega Corp., Madison, Wis.) is added to each of the wells. The cell plates are placed for 30 seconds on an orbital shaker, and luminescence is measured.

Killing of virally infected senescent cells or senescent like cells can be measured in the same manner mutatis mutandis by using senescent cells that are virally infected, comparing results to non-senescent and/or non-infected cells.

### Example 3: Screening a Compound Library for Bcl Antagonists

Discovery of senolytic agents useful for implementation according to this invention was based on the premise that senescent cells can be killed by inhibiting one or more of the Bcl family of regulator proteins that are anti-apoptotic. A molecule with high affinity and selectivity for a Bcl isoform was hypothesized to be effective in inducing apoptosis in senescent cells but not proliferating or non-senescent cells of the same tissue type. Compounds with these properties would be candidates for development as therapeutic agents for clinical medicine.

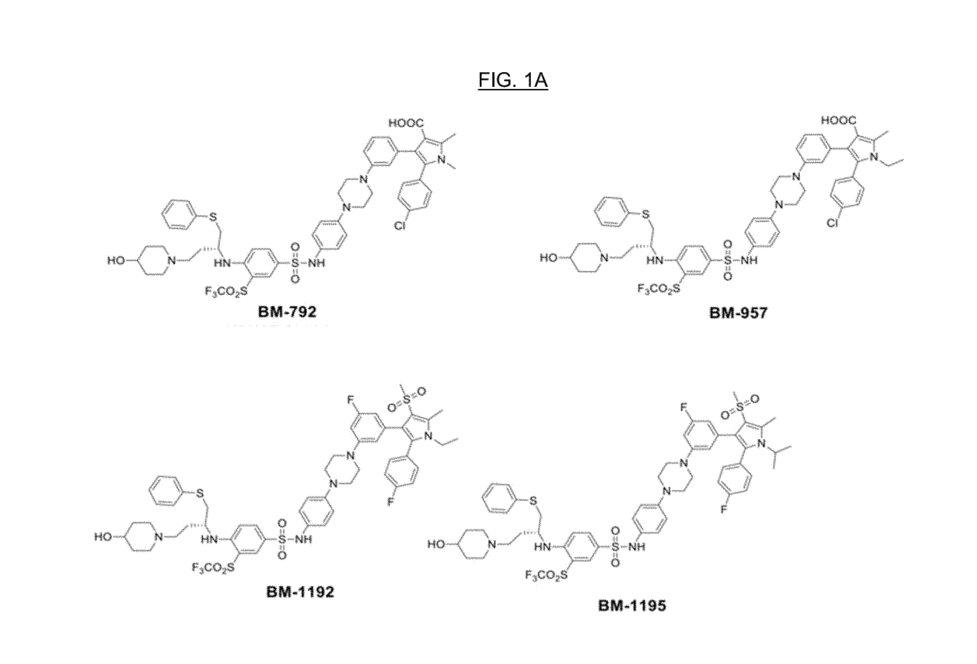

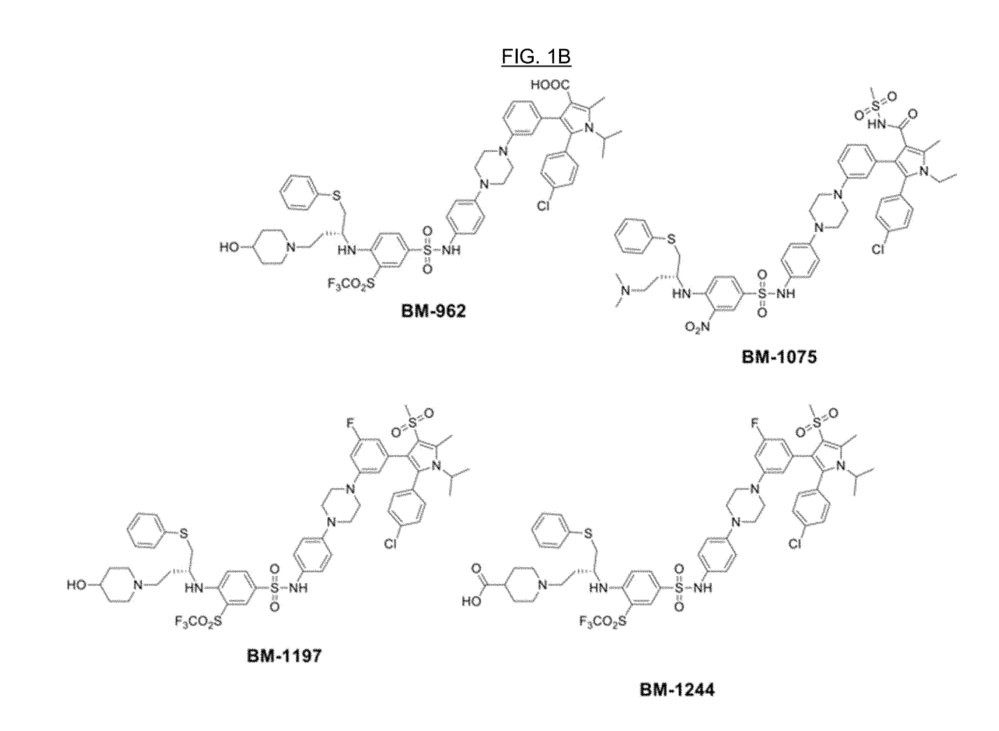

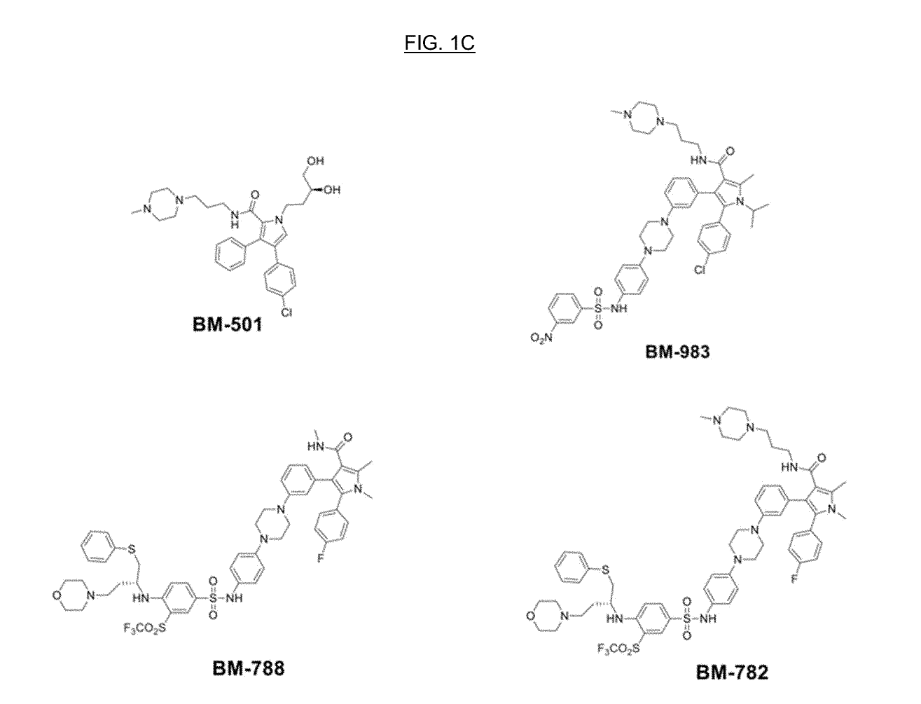

A library that was initially constructed that contained several hundred compounds. Synthesis and use of such library are explained in U.S. Pat. Nos. 8,691,184, 9,096,625, and 9,403,856. The library was initially screened elsewhere for compounds that were able to bind or inhibit Bcl-xL and/or Bcl-2. Fifteen compounds were chosen from the initial screening for further analysis. FIGS. 1A, 1B, and 1C show nine of the fifteen compounds.

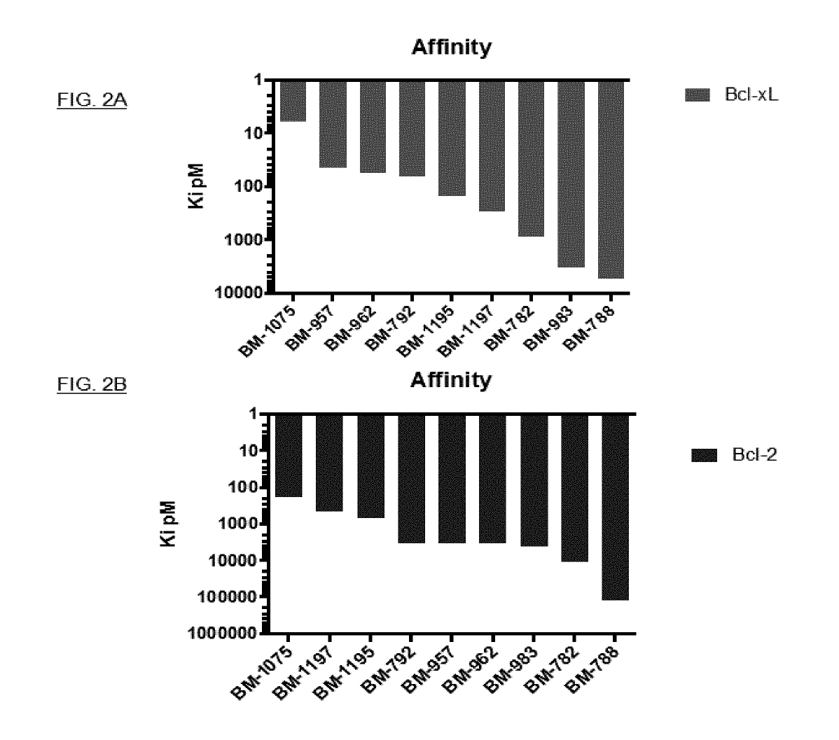

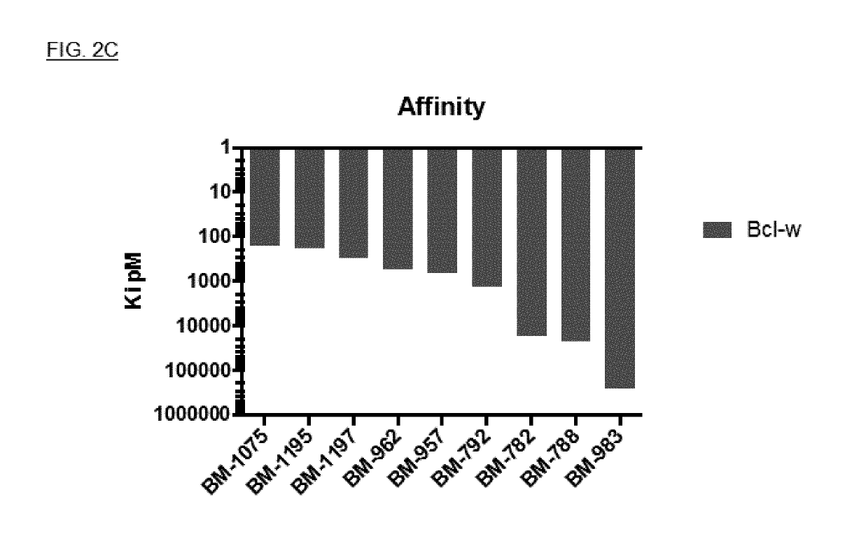

The chosen compounds were further assayed to quantitatively determine the actual affinity for Bcl-xL, Bcl-2, and Bcl-w with a view to identifying candidate senolytic agents for use in treating age related conditions. FIGS. 2A, 2B, and 2C show the results of the binding assay, with those compounds towards the left of each graph having the highest affinity.

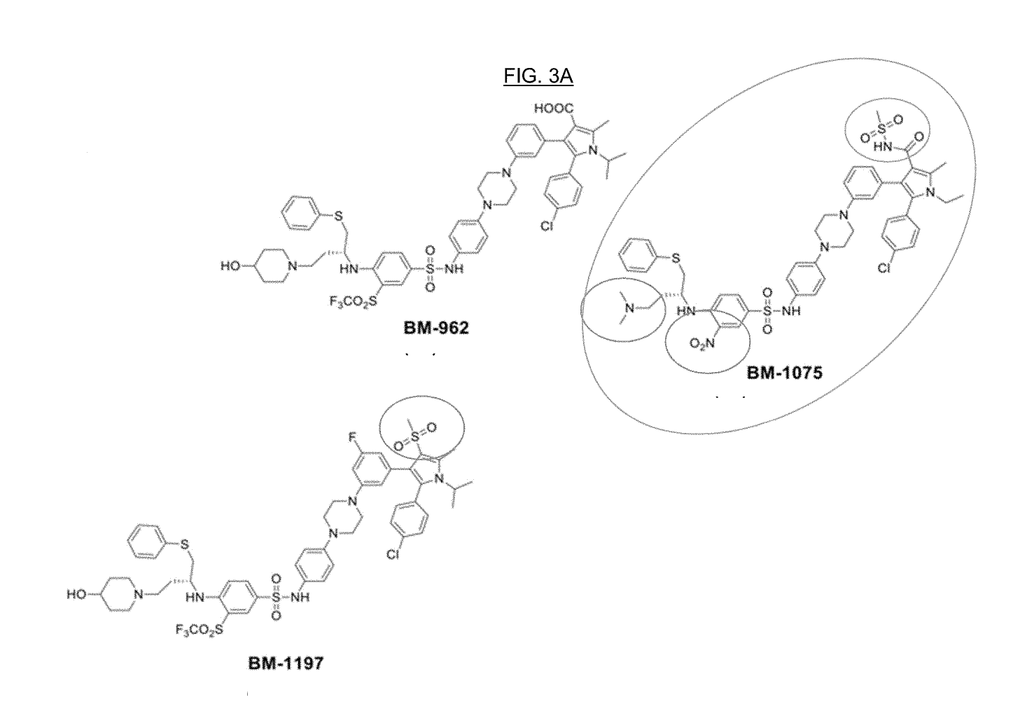

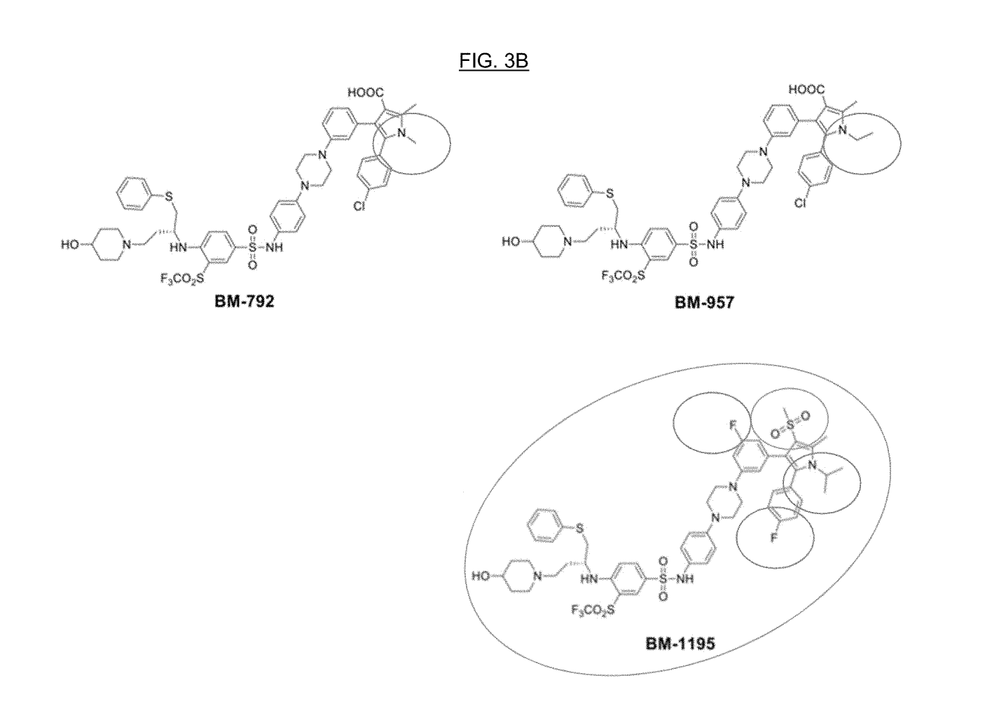

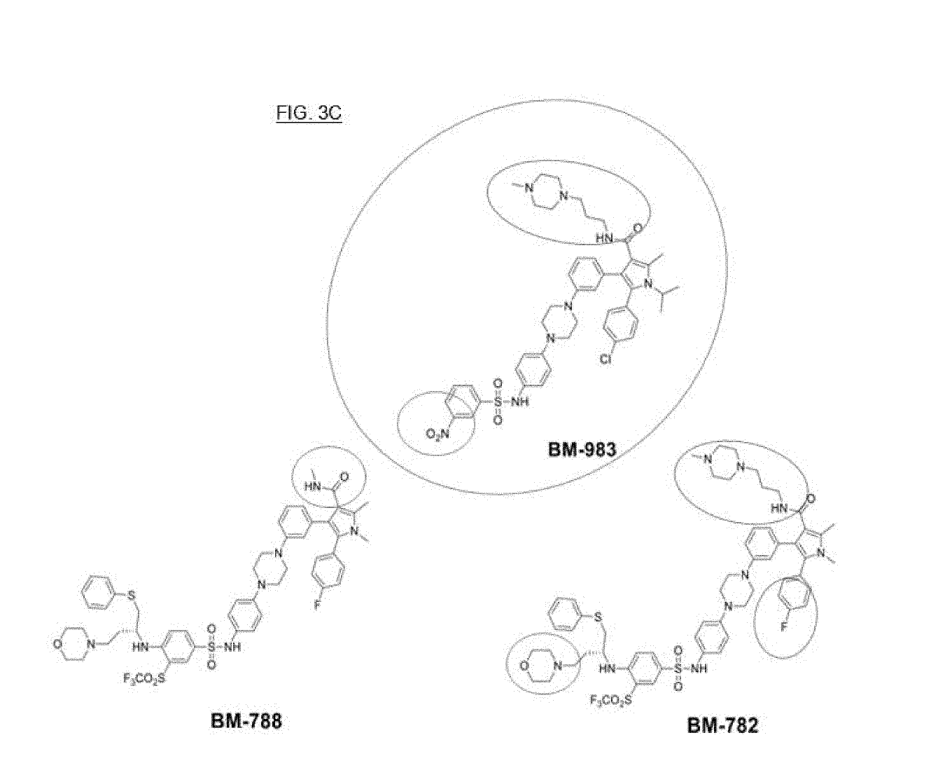

Individual compounds in the library had similar core structures. As shown in FIGS. 3A, 3B, and 3C, the structures of each of the compounds having the most promise were compared with a view to identifying substituents of the molecular structure that were contributing to the desired effect.

### Example 4: Screening High Affinity Bcl Antagonists for Senolytic Potency and Specificity

An ability to bind Bcl regulatory proteins does not necessarily mean that the compound is suitable for inducing apoptosis in a clinical setting. Furthermore, even if the compound is potent, it would not be suitable for use as a therapeutic unless it preferentially kills senescent cells with a high degree of selectivity. Accordingly, compounds in the library showing high Bcl binding affinity were further screened for their ability to kill irradiated fibroblasts, in comparison with replicating fibroblasts or fibroblasts that were quiescent (due to confluence) but not senescent.

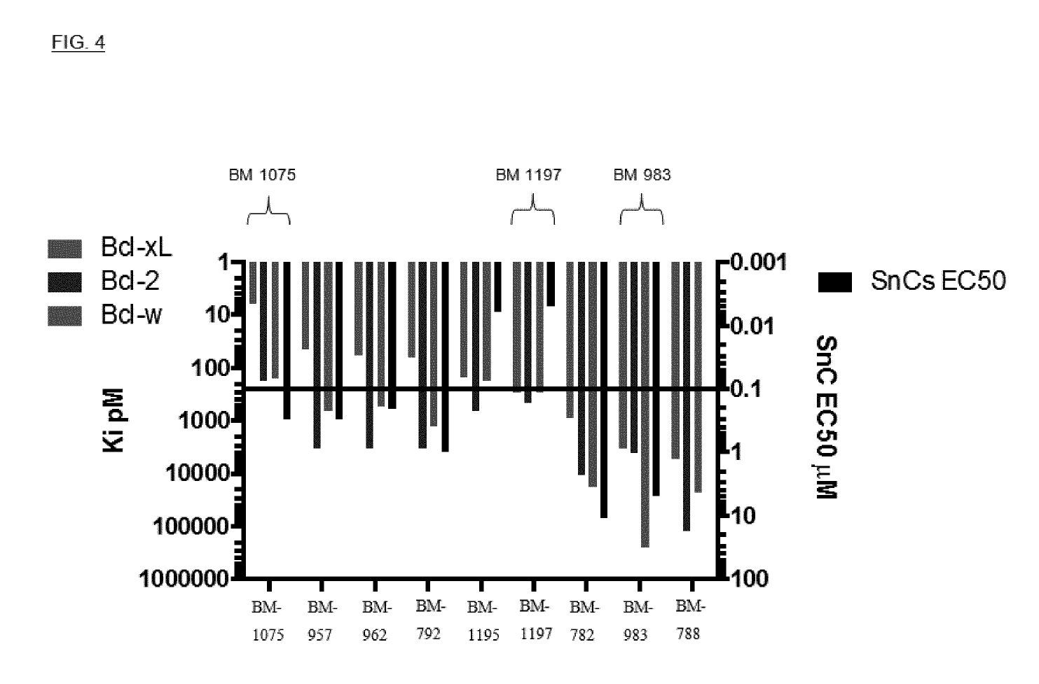

FIG. 4 shows data obtained from the nine model compounds: affinity of binding to each of the Bcl isoforms, and the effective concentration (EC₅₀) for killing senescent fibroblasts (SnCs) in culture. The data are summarized in TABLE 1, below.

| | | | | | Best | |
|---|---|---|---|---|---|---|
| | Molecular | Binding Affinities (Kᵢ ± SD) | | | SnCs EC₅₀ | |
| Original IDs | Weight | Bcl-xL (pM) | Bcl-2 (pM) | Bcl-w (pM) | (μM) | SI |
| BM-501 | 639.1 | NA | NA | NA | NA | NA |
| BM-983 | 967.5 | 3307 | 4037 | >250000 | 5.000 | 1.0 |
| BM-782 | 1274.4 | 885.55 | 10923 | 17694 | 11.000 | 1.3 |
| BM-788 | 1148.2 | 5292 | 121045 | 23132.5 | NA | NA |
| BM-792 | 1165.7 | 64.125 | 3324 | 1295.5 | 1.000 | 3.3 |
| BM-957 | 1179.7 | 45.3 | 3333 | 659.95 | 0.300 | 11.7 |
| BM-962 | 1193.7 | 57.41 | 3389.5 | 538.15 | 0.200 | 18.5 |
| BM-1075 | 1113.7 | 5.7285 | 178.95 | 161.45 | 0.300 | 15.3 |
| BM-1192 | 1215.3 | NA | NA | NA | 0.050 | 60.0 |
| BM-1195 | 1229.3 | 148.565 | 666.55 | 174.7 | 0.006 | 128.7 |
| BM-1197 | 1245.8 | 295 | 447.95 | 291.6 | 0.005 | 366.7 |
| BM-1244 | 1273.8 | 134.52 | 450.75 | 356.75 | 0.300 | 66.7 |
| BM-1261 | 1409.9 | 106.89 | 274.45 | 239.45 | 27.000 | NA |
| BM-1252 | 1395.9 | 81.085 | 135.85 | 123.95 | 6.000 | NA |

The data show that binding to any of the Bcl isoforms with high affinity was not necessarily predictive of an effective senolytic agent. The compounds designated BM-1075, BM-1195, BM-1197, BM-1244, BM-1261, and BM-1252 all had binding affinities (Ki) for the Bcl isoforms that were in the nanomole to picomole range. However, in the assay to determine effective concentration for killing the cells (EC₅₀), some of these molecules, such as BM-1244, BM-1261, and BM-125, were 60 to 5,000-fold less potent than the compounds ultimately chosen for development.

The compounds with the best senolytic activity, BM-1195 and BM-1197, were potent in the nanomole range. There was a wide range of specificity for senescent cells (SI) determined for the various compounds, ranging from 1.0 (non-specific) to over 300. The best compound in terms of both potency and specificity was BM-1197, with BM-1195, BM-1244, BM-1105, and BM-1075 also being of interest.

In accordance with the data, the following deductions were made with respect to the chemical structure. This substructure was at the core of effective compounds:

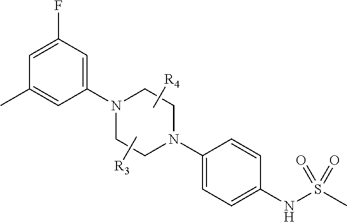

with the R₃, R₄, and F groups being optional. The —SO₂CF₃ in BM-1197 is influential, but could be substituted with groups having similar properties, such as —NO₂ in BM-1075. The —SO₂R′ group is influential, although R′ could be varied from —CH₃ to other short-chain alkyl groups. The aryl —S—C₆H₅ group is also influential, although it could potentially have neutral substituents. With respect to the following part of the structure:

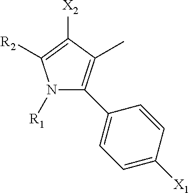

R₁ can be several short-chain alkyl groups, and X₁ can be varied (Cl in BM-1197; F in BM-1195). The following part of BM-1197:

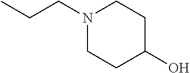

appears to be forgiving in form, with a range of alternative substructures being effective for many purposes of this invention.

These and other deductions lead to the drawing of the generic structure shown earlier as Formula VI and Formula VII.

### Example 5: Efficacy of Compounds in an Animal Model of Osteoarthritis

C57BL/6J mice undergo surgery to cut the anterior cruciate ligament of one rear limb to induce osteoarthritis in the joint of that limb. At week 2 post-surgery, mice receive 2.5 μg of test compound to the operated knee by intra-articular injection, q.d. for 5 days, with a second treatment (2.5 μg test compound-q.d. for 5 days) during week 4 post-surgery. At the end of 4 weeks post-surgery, operated joints of the mice are assessed for function, monitored for markers of inflammation, and undergo histological assessment.

Two control groups of mice are included: One group comprising C57BL/6J mice that undergo a sham surgery (i.e., surgical procedures are followed except for cutting the ACL) and receive intra-articular injections of vehicle parallel to the treated group; and one group comprising C57BL/6J that undergo an ACL surgery and receive intra-articular injections of vehicle.

Function of the limbs are assessed at 4 weeks post-surgery by a weight bearing test to determine which leg the mice favor. The mice are allowed to acclimate to the chamber on at least 3 occasions prior to taking measurements. Mice are maneuvered inside the chamber to stand with 1 hind paw on each scale. The weight that is placed on each hind limb is measured over a 3-second period. At least 3 separate measurements are made for each animal at each time point. The results are expressed as the percentage of the weight placed on the operated limb versus the contralateral unoperated limb.

The function of the limbs are also assessed at 4 weeks post-surgery by hotplate analysis to show sensitivity and reaction to pain stimulus. In brief, a mouse is placed on a hotplate at 55° C. When placed on the hot surface of the plate, mice will lift their paws and lick them (paw-lick response) due to attainment of pain threshold. The latency period for the hind limb response (paw-lick response) is recorded as response time. Histopathology of the proteoglycan layer is also analyzed.

### Example 6: Efficacy of Compounds in an Animal Model of Cardiac Stress Resistance

At 12 months of age, mice are injected three times per week with the test compound, while a control group receives vehicle. At 18 months, subsets of male and female mice are subjected to a cardiac stress test, in which mice are injected with a lethal dose of isoproterenol (680 mg/kg) and the time to cardiac arrest is recorded. The time to cardiac arrest is compared between treated and untreated animals.

### Example 7: Efficacy of Compounds in an Animal Model of Cardiac Stress Resistance

LDL^{-/-}mice from 10 weeks of age are fed a high fat diet having 42% calories from fat beginning at Week 0 until Week 12.5. The mice are then switched to a normal chow diet. Mice are treated with a test compound or a vehicle from week 12.5 over the next 100 days, with each treatment cycle comprising 5 days of test compound (25 mg/kg intraperitoneally daily) and 14 days off. At the end of the 100 day treatment period, mice are sacrificed, plasma and tissues are collected, and atherosclerosis is quantitated. Descending aortas are dissected and stained with Sudan IV to visualize the plaque lipids. The percentage of the aorta covered in plaques is measured by area, and is compared between the treated and untreated animals.

### Example 8: Efficacy of Compounds in Animal Models of Pulmonary Disease

To assess the efficacy of compounds in treating pulmonary diseases, a model of bleomycin-induced injury is used. In this model, mice develop lung fibrosis within 7-14 days after bleomycin treatment.

Bleomycin is administered to anesthetized 6-8 week-old mice by intratracheal aspiration (2.5 U/kg of bleomycin in 50 μL PBS) using a microsprayer syringe. Control mice are administered saline. The day following bleomycin treatment, test compound (25 mg/kg in PBS) or vehicle is administered. Mice are treated via intraperitoneal injection for 5 consecutive days, followed by 5 days of rest, followed by a second treatment cycle of 5 consecutive days. Untreated mice receive an equal volume of vehicle. At 7, 14, and 21 days post-bleomycin treatment, lung function is assessed by monitoring oxygen saturation using the MouseSTAT™ PhysioSuite pulse oximeter (Kent Scientific). Animals are anesthetized with isoflurane (1.5%) and a toe clip is applied. Mice are monitored for 30 seconds and the average peripheral capillary oxygen saturation (SpO₂) measurement over this duration is calculated.

At 21 days post-bleomycin treatment, airway hyper-reactivity (AHR) of mice is examined. AHR of mice is measured by methacholine challenge while other parameters of lung function (airway mechanics, lung volume and lung compliance) are determined using a SCIREQ™ flexiVent ventilator. While under ketamine/xylazine anesthesia and subjected to cannulation of the trachea via a tracheostomy (19Fr blunt Luer cannula), airway resistance (elastance) and compliance of mice are assessed at baseline and in response to increasing concentrations of methacholine (0 to 50 mg/mL in PBS) delivered via nebulization (AeroNeb). Animals are maintained at 37° C., and while under muscle paralysis (pancuronium); airway function is measured by using the FlexiVent™ ventilator and lung mechanics system (SCIREQ, Montreal, Quebec, Canada).

Mice are euthanized by intraperitoneal injection of pentobarbital. Bronchoalveolar lavage (BAL) fluids and lungs is obtained and analyzed. Hydroxyproline content of lungs is measured and quantitative histopathology is performed.

In a second animal model for pulmonary diseases (e.g., COPD), mice are exposed to cigarette smoke. The effect of a test compound on the mice exposed to smoke is assessed by lung function and histopathology.

Six week-old mice are chronically exposed to cigarette smoke from a Teague TE-10 system, an automatically-controlled cigarette smoking machine that produces a combination of side-stream and mainstream cigarette smoke in a chamber, which is transported to a collecting and mixing chamber where varying amounts of air is mixed with the smoke mixture. Mice receive a total of 6 hours of cigarette smoke exposure per day, 5 days a week for 6 months. Each lighted cigarette is puffed for 2 seconds and once every minute for a total of 8 puffs, with the flow rate of 1.05 L/min, to provide a standard puff of 35 cm³. The smoke machine is adjusted to produce a mixture of side stream smoke (89%) and mainstream smoke (11%) by smoldering 2 cigarettes at one time. The smoke chamber atmosphere is monitored for total suspended particulates (80-120 mg/m³) and carbon monoxide (350 ppm). Beginning at day 7, mice are treated with test compound or vehicle (3× per week) (5 consecutive days of treatment followed by 16 days off drug, repeated until the end of the experiment), respectively. An equal number of mice received the corresponding vehicle.

After two months of cigarette smoke exposure, lung function is assessed by monitoring oxygen saturation using the MouseSTAT PhysioSuite pulse oximeter (Kent Scientific). Animals are anesthetized with isoflurane (1.5%) and the toe clip is applied. Mice are monitored for 30 seconds and the average peripheral capillary oxygen saturation (SpO₂) measurement over this duration is calculated. At the end of the experimental period, airway hyper-reactivity (AHR) of mice to methacholine challenge using a SCIREQ flexiVent ventilator and lung mechanics system is examined as described above. After AHR measurement, mice are killed by intraperitoneal injection of pentobarbital for in-depth analysis of lung histopathology. Briefly, lungs are inflated with 0.5% low-melting agarose at a constant pressure of 25 cm. Lungs are fixed in 10% buffered formalin and embedded in paraffin. Sections (5 μm) are stained with hematoxylin and eosin. Mean alveolar diameter, alveolar length, and mean linear intercepts are determined by computer-assisted morphometry with Image Pro Plus™ software (Media Cybernetics).

The several hypotheses presented in this disclosure provide a premise by way of which the reader may understand the invention. This premise is provided for the enrichment and appreciation of the reader. Practice of the invention does not require detailed understanding or application of the hypothesis. Except where stated otherwise, features of the hypothesis presented in this disclosure do not limit application or practice of the claimed invention. For example, except where the elimination of senescent cells is explicitly required, the compounds of this invention may be used for treating the conditions described regardless of their effect on senescent cells.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed.

1. A method of treating a pulmonary condition mediated by senescent cells,
the method comprising administering in or around lung tissue of the subject a pharmaceutical composition that includes a compound having a chemical structure that comprises Formula (VII) as shown below, or a phosphorylated form thereof: 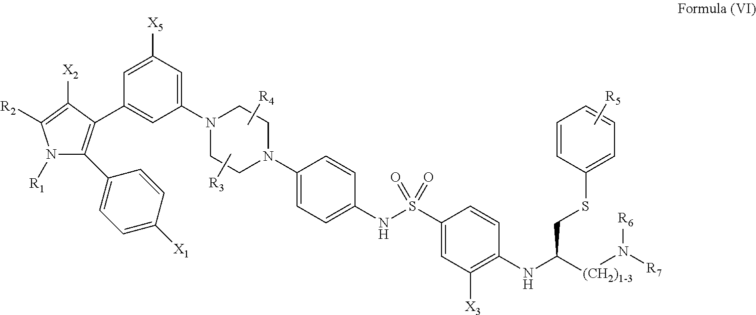
wherein:
R₁ and R₂ are independently C₁ to C₄ alkyl
R₃, R₄ and R₅ are independently —H or —CH₃;
R₆ and R₇ are independently alkyl or heteroalkyl, and are optionally cyclized;
X₁ is —F, —Br, or —OCH₃;
X₂ is —SO₂R′ OR —CO₂R′, where R′ is —H, —CH₃, or —CH₂CH₃;
X₃ is —SO₂CF₃ or —NO₂; and
X₅ is —F, —Br, —Cl, —H, or —OCH₃.

2. The method of claim 1, wherein the compound has a chemical structure that comprises Formula (VI) as shown below, or a phosphorylated form thereof. 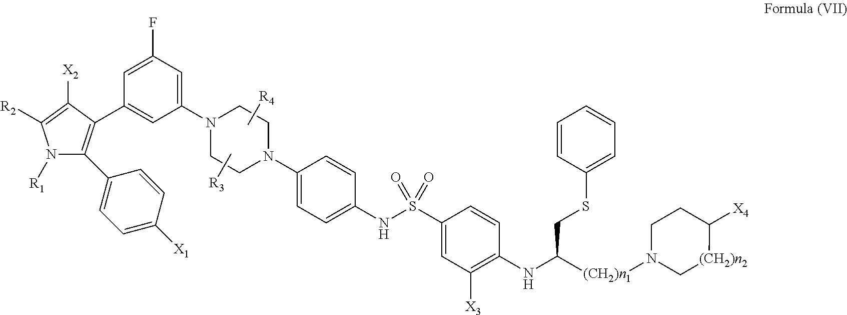
wherein:
R₁ and R₂ are independently C₁ to C₄ alkyl;
R₃ and R₄ are independently —H or —CH₃;
R₆ and R₇ are independently alkyl or heteroalkyl, and are optionally cyclized;
X₁ is —F, —Br, or —OCH₃;
X₂ is —SO₂R′ OR —CO₂R′, where R′ is —H, —CH₃, or —CH₂CH₃;
X₃ is —SO₂CF₃ or —NO₂;
X₄ is —OH or —COOH; and
n₁ and n₂ are independently 1, 2, or 3.

3. The method of claim 2, wherein the compound has one or more of the following features in any combination:
R₁ is isopropyl;
R₂ is methyl;
R₃ is —H;
R₄ is —H;
X₁ is —Cl;
X₂ is —SO₂CH₃;
X₃ is —SO₂CF₃;
X₄ is —OH;
n₁ is 2; and
n₂ is 2.

4. The method of claim 1, wherein the compound is a phosphorylated form of Formula (VI).

5. The method of claim 1, wherein the compound is a non-phosphorylated form of Formula (VI).

6. The method of claim 2, wherein the compound is a phosphorylated form of Formula (VII).

7. The method of claim 2, wherein the compound is a non-phosphorylated form of Formula (VII).

8. The method of claim 3, wherein the compound has all of said features.

9. The method of claim 1, wherein the pulmonary condition is idiopathic pulmonary fibrosis (IPF).

10. The method of claim 1, wherein the pulmonary condition is chronic obstructive pulmonary disease (COPD).

11. The method of claim 2, wherein the pulmonary condition is idiopathic pulmonary fibrosis (IPF).

12. The method of claim 2, wherein the pulmonary condition is chronic obstructive pulmonary disease (COPD).

13. The method of claim 1, wherein the compound is administered by inhalation as an aerosol.

14. The method of claim 2, wherein the compound is administered by inhalation as an aerosol.

15. The method of claim 1, wherein the compound is administered in a plurality of treatment cycles, wherein each treatment cycle independently comprises a treatment course followed by a non-treatment interval of at least 2 weeks

16. The method of claim 1, wherein the compound is administered as a single dose followed by a non-treatment interval of at least 2 months.

17. A pharmaceutical composition for treatment of a pulmonary condition,
wherein the composition is a pharmaceutical formulation that contains a concentration of the compound shown in Formula (VI) or Formula (VII),
wherein the formulation is configured for aerosol administration of the composition into a lung of a subject in need thereof, and
wherein the formulation of the composition and the concentration of the compound in the composition configure the composition to be effective in treating the pulmonary condition by decreasing the severity of one or more signs or symptoms of the condition when administered by aerosol into the lung of the subject.

18. The composition of claim 17, wherein the pulmonary condition is idiopathic pulmonary fibrosis (IPF).

19. The composition of claim 17, wherein the pulmonary condition is chronic obstructive pulmonary disease (COPD).

20. The composition of claim 18, packaged with information for administration for use of the composition for treatment of the pulmonary condition.
